(19) United States
(12) Patent Application Publication
(10) Pub. No.: US 2021/0194600 A1
(43) Pub. Date: Jun. 24, 2021

(54) MOLECULAR COMMUNICATION AND MOLECULAR SIGNALLING- A SYSTEM AND METHOD

(71) Applicants: AbdulAziz Al-Helali, Toronto (CA); Ben Liang, Toronto (CA); Alfaisal University, Riyadh (SA)

(72) Inventors: AbdulAziz Al-Helali, TORONTO (CA); Ben Liang, TORONTO (CA); Nidal Nasser, RIYADH (SA)

(73) Assignees: Alfaisal University, Riyadh (SA); AbdulAziz Al-Helali, Toronto (CA); Ben Liang, Toronto (CA)

(21) Appl. No.: 16/720,908

(22) Filed: Dec. 19, 2019

(51) Int. Cl. H04B 10/90; A61N 1/372; A61M 31/00

(52) U.S. Cl. CPC H04B 10/90; A61B 34/30; A61M 31/002; A61N 1/37217

(57) ABSTRACT

A molecular communication system in body (MoCoBo) and a molecular signaling method (MoSiMe) for communicating information via molecules in a body of an organism with a circulatory system are disclosed. The MoCoBo system includes a transmitter configured to release signaling molecules in the body in a controlled manner to modulate information, and a receiver for receiving data by measuring the quantity of molecules in the body to infer the encoded information, and a communication channel which is a body of an organism with a circulatory system used as the medium through which the signaling molecules travel from the transmitter to the receiver. The MoSiMe method exploits the bodily absorption, distribution, metabolization, and excretion processes to create a signal across the body by controlling the amount of signaling molecules and the time of release so that the receiver will be able to detect and extract the encoded information within it.

### FIELD OF INVENTION

The present invention relates generally to molecular communications, and more specifically, to sending information wirelessly inside the body of an organism with a circulatory system such as a human or animal.

### BACKGROUND

Implanted devices are electronic devices that are installed inside the body for medical purposes. They are designed to stay in the body for long periods up to few years in some applications. They can be used for treatment or diagnostic purposes. Heart pacemakers, insulin pumps, and neurostimulators are some examples of implanted medical devices. These devices are equipped with communication and networking functionalities that allow them to provide sophisticated features such as reporting measurements or alarms in real time or changing their settings or configuration without removing them from the body.

Currently, implanted devices communicate either percutaneously or wirelessly. In first mechanism, wires are run through the skin of the patient and connected to the implanted device from one side and the other side is connected to interface at the skin to allow external devices to be hooked to. Although this method provides fast, reliable, secure, low cost, low power consumption, its use is limited to testing and prototyping purposes because of two main problems. First, the interface creates openings in the skin that becomes subject to infections and disclose the use of an implanted device. Second, the wires restrict the movement of the patient.

On the other hand, wireless communication provides a convenient and invisible way of communication with implanted devices which make it used widely. Implanted devices can be equipped with active or passive components that realize the wireless communication link. The active components have active electronic circuits that requires a battery to power the device to transmit or receive a wireless signal. This forces designers to increase implanted size to accommodate a large battery that can work for years. In contrast, the passive components use induction from a device outside the body as a power source. Although they do not need a battery to run, they need to have large antennas and need to be placed near the skin to increase power transfer efficiency during the induction process. Therefore, it is not suitable for devices that should be implanted deeply inside the body. Since the active and passive methods use electromagnetic waves for signaling, they are vulnerable to security and privacy attacks due to interference and information leakage. Using specialized devices, malicious users could target those devices from a remote location to gain access, reconfigure, disable, or eavesdrop on them. There is a need for a safer yet efficient communication system to enable wireless communication with such devices.

### SUMMARY

This invention relates to molecular communication system and method that transfer information via the body of an organism with a circulatory system secretly, safely, and passively with low power consumption while transmitting the signal. One application to mention but not limited to is to enable communication with and between implanted devices in the body.

In one aspect of the invention, a molecular communication system in body (MoCoBo) for communicating information via molecules in a body of an organism with a circulatory system is provided comprising a transmitter which is configured to release signaling molecules in the body in a controlled manner to modulate information, and a receiver for receiving data by measuring the quantity of molecules in the body to infer the encoded information and a communication channel which is a body of an organism with a circulatory system used as the medium through which the signaling molecules travel from the said transmitter to the said receiver. In one embodiment, the invention relates to enabling communication with and between implanted medical devices in an organism.

Also, in one embodiment, the transmitter works from inside of the body. Also, in one embodiment, the transmitter works from out of the body by attaching it from outside in the form of a patch or connected using a needle or a cannula. Also, in one embodiment, the receiver is an implanted electronic device or a nanorobot, an artificial organism, biologically modified organism, or non biologically modified organism that responds to the transmitted molecular signal. Also, in one embodiment, the transmitter is a passive transmitter that could deliver molecular signals without electrically powered parts utilizing physical and chemical properties of the body. Also, in one embodiment, the transmitter is in the form of a pill taken orally comprises a compartment or a plurality of compartments where each compartment may contain a solid or liquid form of signaling molecules or combination of both. The compartments may be made from membranes or materials that has different biodegradability or made from the same material with different thickness so that the dissolution time of one compartment is different than the other. Also, in one embodiment, the transmitter is in the form of a patch comprises of a compartment or a plurality of compartments where each compartment may contain a solid or liquid form of signaling molecules or combination of both. The compartments control the release of signaling molecules into the body by being equipped with membranes or materials that has different biodegradability or made from the same material with different thickness or have different size apertures so that the release time of one compartment is different than the other. The signaling molecules may be carried from the compartments and injected into the body via micro needles. Also, in one embodiment, the transmitter is in the form of an active pill taken orally comprises a microcontroller, power source, signaling molecules reservoir, a releasing mechanism, and an aperture.

Also, in one embodiment, the transmitter may be placed inside a box comprises an infusion set that has a cannula. The cannula is inserted through the skin into the body and may have a patch to hold the cannula in place while delivering the signaling molecules to the body.

In another aspect of the invention, a molecular signaling method (MoSiMe) is provided, comprising the steps (a) injecting the signaling molecules into the body by a transmitter to modulate a symbol, (b) stopping the injection of the signaling molecules into the body by the transmitter, (c) Waiting till signaling molecules concentration is maintained the same or decrease over a period of time then go to step (a), otherwise stop.

In one embodiment of the method, the transmitter controls the amount and release time of signaling molecules using one or all parameters of ADME processes to inject them into the body for creating a MoSiMe signal sufficient for the receiver to detect and extract the encoded information within it. Also, in one embodiment the transmitter could use more than one type of signaling molecule to transmit more than one symbol at the same time. Other features will be apparent from the accompanying drawings and from the detailed description that follows.

### BRIEF DESCRIPTION OF DRAWINGS

Example embodiments are illustrated by way of example only and not limitation, with reference to the figures of the accompanying drawings, in which like references indicate similar elements and in which:

FIG. 1 is a diagram showing one configuration of molecular communication in body (MoCoBo) system according to an embodiment of the present invention.

FIG. 2 is a diagram showing an example of the concentration profile over time of signaling molecules administered to the body.

FIG. 3 is a process flow chart illustrating steps of molecular signaling method (MoSiMe) according to an embodiment of the present invention

FIG. 4 is a diagram showing an example of a molecular signal (MS) modulating a stream of bits using two levels of concentration according to an embodiment of the present invention.

FIG. 5 is a diagram showing system-level model for molecular communication in body (MoCoBo) according to an embodiment of the present invention.

FIG. 6 is a process flow chart illustrating steps of an example method used by the transmitter according to an embodiment of the present invention.

FIG. 7 is a process flow chart illustrating steps of an example method used by the receiver according to an embodiment of the present invention.

FIG. 8 is a diagram showing an example signaling molecules doses that generates a molecular signal (MS) for modulating a stream of bits (11101010011) according to an embodiment of the present invention.

FIG. 9 is a diagram showing an example of an analytical and experimental normalized impulse response for the intravenous and extravascular administration according to an embodiment of the present invention.

FIG. 10 is a diagram showing an example of a modeled and an experimental molecular signal wave in body captured by a receiver for input signals given to the body by intravenous and extravascular administrations according to an embodiment of the present invention.

FIG. 11 is a diagram showing an example of a modeled and an experimental recovered input signal at the receiver for intravenous administrations according to an embodiment of the present invention.

FIG. 12 is a diagram showing an example of a modeled and an experimental recovered input signal at the receiver for extravascular administrations according to an embodiment of the present invention.

FIG. 13 is a diagram showing examples of different types of MoCoBo transmitters.

Other features of the present disclosure will be apparent from the accompanying drawings and from the detailed description of embodiments that follows.

### DETAILED DESCRIPTION

In this disclosure a Molecular Communication system in the Body (MoCoBo) and an alternative communication method called Molecular Signaling Method (MoSiMe) is described. In this system and method signaling molecules are used instead of electromagnetic waves to provide better privacy and security compared to existing systems. The signaling molecules are released in, and confined to, the body fluids which provide high security against eavesdropping, particularly when compared to electromagnetic waves. In addition, hijacking the communication link cannot take place without noticeable physical contact with the body which the patient would be aware of in most cases and thus be capable of preventing it. Moreover, unlike electromagnetic waves that are power hungry and risk harming body cells, this method is biocompatible and power efficient. We propose a novel system and method of communication using a new way of wireless signaling using molecules. The proposed communication method takes advantage of the bodily processes such as absorption, distribution, metabolism, and excretion (ADME).

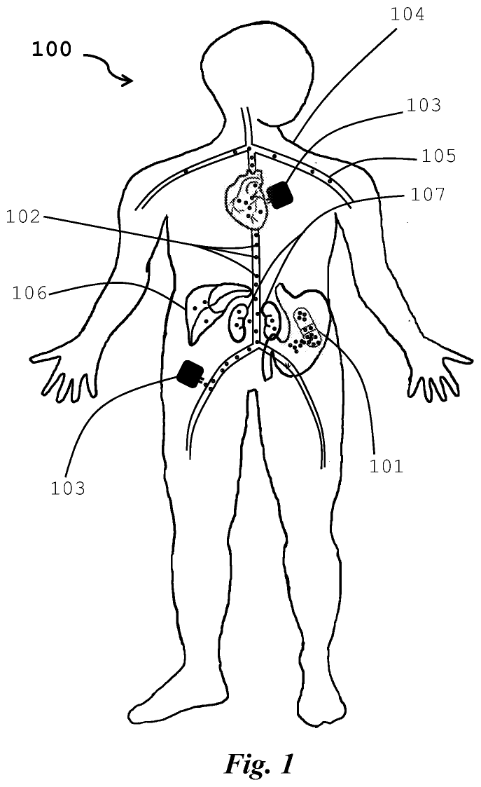

FIG. 1 is a diagram showing one configuration of molecular communication system in body 100 (MoCoBo) according to an embodiment of the present invention. The MoCoBo system 100 comprises a transmitter 101 or a set of transmitters in another embodiment, signaling molecules 102, a receiver 103 or a set of receivers in another embodiment, communication channel 104 which is a body of an organism with a circulatory system 105. In this example, the transmitter 101 is in the form of a pill that is taken orally. The transmitter 101 is configured to release signaling molecules 102 in a controlled manner to modulate information so that the receiver 103 can detect it and infer the encoded information. The body 104 of an organism with a circulatory system 105 is the communication channel that is used as the medium through which the signaling molecules travel from the transmitter 101 to the receiver 103. The body 104 and channel terms will be used interchangeably in the following text.

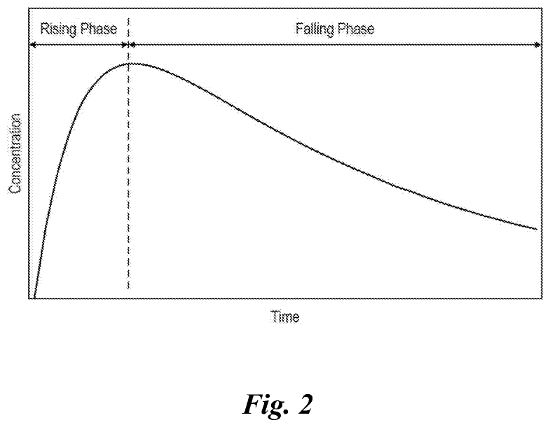

FIG. 2 is a diagram showing an example of the concentration profile over time of signaling molecules administered to the body. The signaling molecules FIG. 1 (102) distribution across the body is affected by body 104 processes such as absorption, distribution, metabolization and excretion (ADME). When signaling molecules are released into the body 104, the body ADME processes change the signaling molecules concentration over time which generates a signal across the body that can be detected at the receiver FIG. 1 (103). The signal amplitude changes in proportion to the concentration of signaling molecules in the body over time. The signal has two phases: the rising phase and falling phase. The rising phase starts after injecting signaling molecules into the body 104 as the concentration starts to increase with a rate proportional to how fast the signaling molecules get absorbed by the body and reach the systolic system in a bodily process known as absorption. As the signaling molecules enter the blood system, the distribution process start where the blood carries the signaling molecules and distribute them all over the body which leads to an increase in the signaling molecules concentration. However, the signaling molecules gets metabolized (i.e. broken down into other compounds) and excreted as they go through organs such as the liver, kidneys, and sweat glands. The concentration keeps increasing even though the metabolization and excretion processes reduce it because the absorption process happens at a faster rate. When the injection of signaling molecules into the body stops, the absorption rate reduce and the metabolization and excretion processes rate becomes higher. Therefore, the falling phase start and leads to a decline in the signaling molecules' concentration.

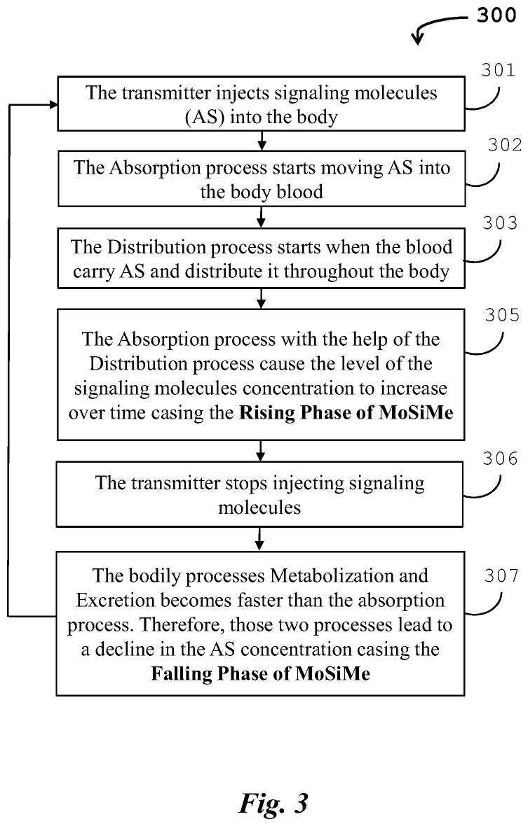

FIG. 3 is a process flow chart illustrating steps 300 of molecular signaling method (MoSiMe) according to an embodiment of the present invention. The method exploits the bodily ADME process to create a signal across the body by controlling the amount of signaling molecules released and the time of release. At step 301 the transmitter injects signaling molecules into the body. At step 302 The Absorption process starts moving signaling molecules into the body blood. Then, the distribution process starts when the blood carry signaling molecules and distribute them throughout the body at step 303. At step 305 the absorption and distribution processes cause the level of the signaling molecules concentration to increase over time casing the Rising Phase of MoSiMe as shown in FIG. 2. At step 306 the transmitter stops injecting signaling molecules into the body. At step 307 the bodily processes metabolization and excretion becomes faster than the absorption process. The metabolization which is mostly done in the liver and excretion which mostly done by the kidneys eliminate signaling molecules out of the body. Therefore, the metabolization and excretion processes lead to a decline in the signaling molecules concentration casing the Falling Phase of MoSiMe as shown in FIG. 2. Then, the signaling method process goes to step 301 if there are more signals to be sent. The transmitter controls the amount and release time of signaling molecules injection into the body to shape the MoSiMe signal in a way that the receiver will be able to detect and extract the encoded information within it. The amount of signaling molecules and release time are found by examining the ADME processes rates.

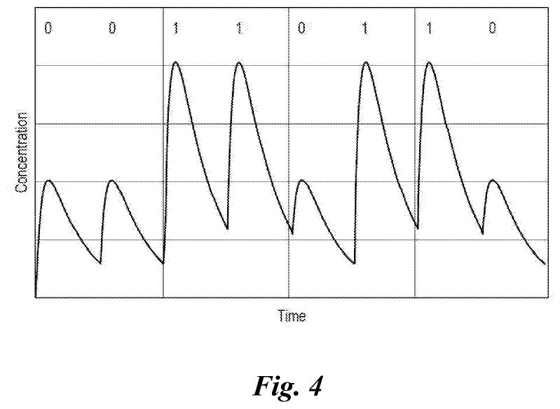

FIG. 4 is a diagram showing an example of a molecular signal (MS) modulating a stream of bits using two levels of concentration (C₀ and C₁) according to an embodiment of the present invention. In this example, the transmitter wants to modulate a MS that encodes symbols that represents bits 0 and 1. To encode a zero, the transmitter injects signaling molecules till the concentration reaches C₀ level, then stops for some time till the concentration level drops before it starts modulating another symbol. On the other hand, if the transmitter wants to encode a one, injects signaling molecules till the concentration reaches C1 level. For example, to modulate a stream of bits such as 001101110, the transmitter injects signaling molecules till the concentration reaches C₀ to modulate a zero. After that it stops till the concentration level drops, then it resumes inject molecules till the concentration reaches C₀ to modulate the second zero. After that it stops till the concentration level drops, then it resumes inject molecules till the concentration reaches C₁ to modulate the first one. After that it stops till the concentration level drops, then it resumes inject molecules till the concentration reaches C₁ to modulate the second one. The process continues till all the bits are modulated.

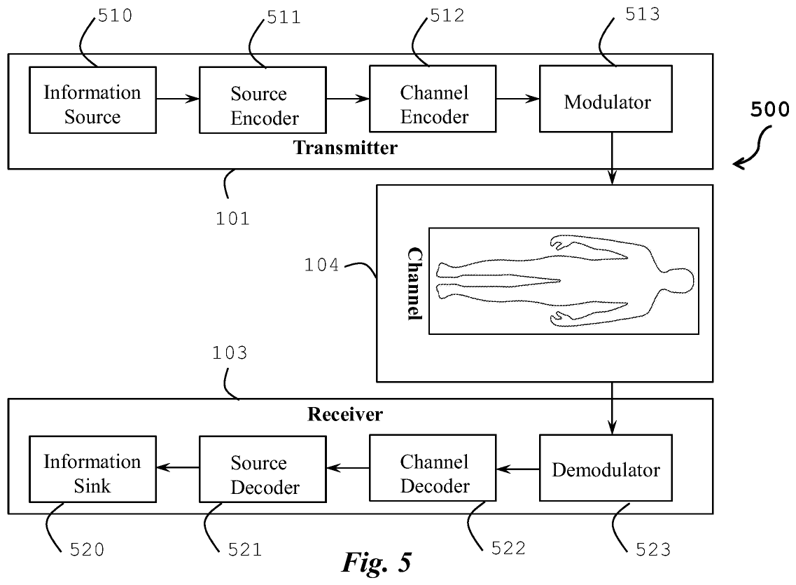

FIG. 5 is a diagram showing system-level model for molecular communication in body (MoCoBo) 500 according to an embodiment of the present invention. In this example, the MoCoBo system 500 comprises a transmitter 101, a channel 104, and a receiver 103 like any traditional communication system. However, the MoCoBo system 500 differs in using the body 104 as the communication channel and employing a new molecular signaling method (MoSiMe) for information signaling which requires changes to the design of the traditional communication system components.

From the logical design point of view, the MoCoBo transmitter 101 components are similar to any traditional transmitter components that are known to a person skilled in the art. In this example, the transmitter 101 may comprise of an information source 510, a source encoder 511, a channel encoder 512, and a modulator 513. The information source 510 has a set of messages which the transmitter 101 wants to send to the receiver 103. The source encoder 511 takes a message and converts it to its binary representation and passes it to the channel encoder 512 where extra bits are added for error detection and correction. The channel encoder 512 passes the generated streams of bits to the modulator 513 which modulate them into the channel 104.

The channel 104 is the communication channel, such as but not limited to, the human body in this example but it could be a body of any organism that has a circulatory system. The channel 104 carries the signaling molecules FIG. 1 (102) from the transmitter's side to the receiver's side.

The receiver 103 can be, but not limited to, any implanted electronic device or a nanorobot, an artificial organism, biologically modified organism, or non biologically modified organism that responds to the transmitted signal that are known to a person skilled in the art. From the logical design point of view, the MoCoBo receiver 103 components are similar to any traditional receiver components that are known to a person skilled in the art. In this example, the MoCoBo receiver 103 may comprise of a demodulator 523, channel decoder 522, source decoder 521, and information sink 520.

In this example, the receiver 103 is assumed to be an implanted electronic medical device such as but not limited to, a pacemaker that may has a microcontroller, power source, and a bio sensor. The microcontroller detects signals received by the biosensor and estimates the channel symbols at the demodulator 523. Then, the receiver 103 runs the channel decoder 522 to detect and correct errors in the transmitted stream of bits. After that, the source decoder 521 predicts the message sent based on the estimated stream of bits and forwards it to the information sink 520.

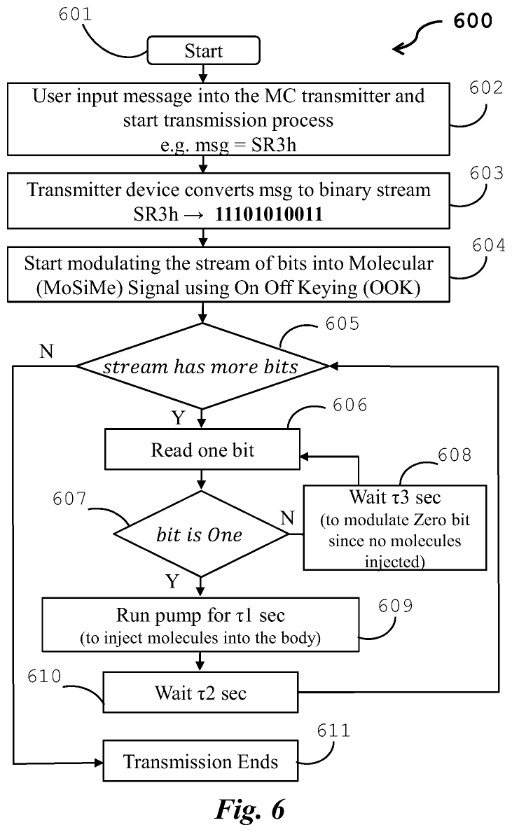

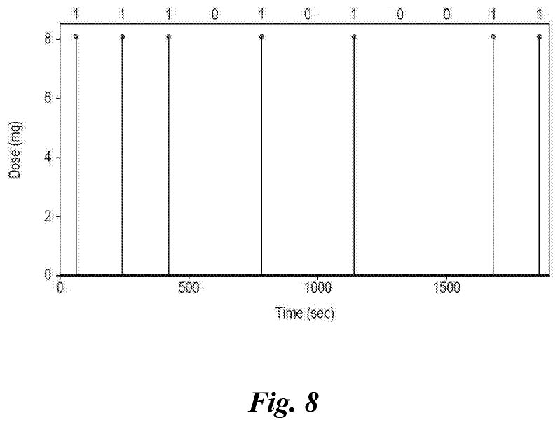

FIG. 6 is a process flow chart illustrating steps 600 of an example method used by the transmitter according to an embodiment of the present invention. The user starts the transmitter at step 601 and enters a message in the transmitter using, but not limited to, an input device such as a keypad at step 602. In this example, the message could be a command that changes the running time to 3 hours and written as SR3h. Then, at step 603 the user selects transmitting the command and the transmitter takes the command and converts it to its equivalent binary stream which could be “11101010011”. At step 604, the transmitter uses a modulation scheme such as, but not limited to, the on off keying (OOK) to create a MoSiMe signal. At step 605, if the stream has more bits, a bit is read at step 606, otherwise, it moves to step 611 and ends the transmission. At step 607, if the bit is one go to step 609 and inject signaling molecules into the body by running a pump for τ1 seconds. Other alternative molecule injection mechanisms are possible and can be used which are apparent to a person skilled in the art. At step 610, the transmitter stops injecting signaling molecules, in this example, by means of stopping the pump for τ2 seconds before picking the second bit. On the other hand, if the bit is zero at step 607, the transmitter moves to step 608 and does not inject signaling molecules to the body, in this example, by keeping the pump off and waits for τ3 seconds, then, it moves to step 605. The process continues till all bits are transmitted where the transmitter moves from step 605 to 611 and ends the transmission. The values for τ1, τ2, and τ3 are selected based on the ADME process rates so that the receiver can detect molecular signal and infer the sent message. An example of a modulated input signal sent using the OOK is shown in FIG. 8.

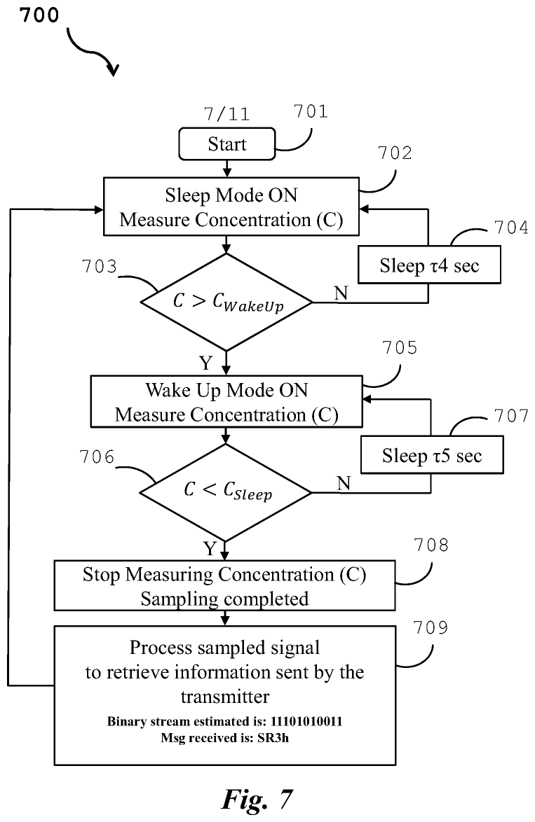

FIG. 7 is a process flow chart illustrating steps 700 of an example method used by the receiver according to an embodiment of the present invention. Once the receiver start 701, it stays on and measures the concentration of the signaling molecules while switching between the sleep and wake up modes. The receiver enters the sleep mode 702 after starting and measures the concentration of the signaling molecules (C). At step 703, it checks if C is less than a predefined concentration level C_WakeUp, it goes to step 704 and sleeps for τ4 seconds before it returns to step 702 and takes another measurement. However, at step 703, if C is greater than C_WakeUp, it assumes that the transmitter is transmitting and moves to step 705 where it changes to wake up mode and measures the signaling molecules concertation. At step 706, if C is greater than a predefined concentration level C_Sleep, it goes to step 707 and waits for τ5 seconds. Then, it returns to step 705 and take another measurement of C. Ideally, τ5 should be much smaller than τ4 to enable the receiver to measure more frequently. In this example, the measured concentration values are stored to be processed later to infer the information send by the transmitter. At step 706, if C is less than C_Sleep, the receiver assumes that the transmitter has finished transmitting and moves to step 708. At step 708, the receiver stops measuring C and moves to step 709. At step 709, the receiver process sampled signal to retrieve information sent by the transmitter. Then, it returns to sleep mode at step 702. Examples of the received sampled signal curves are shown in FIG. 10.

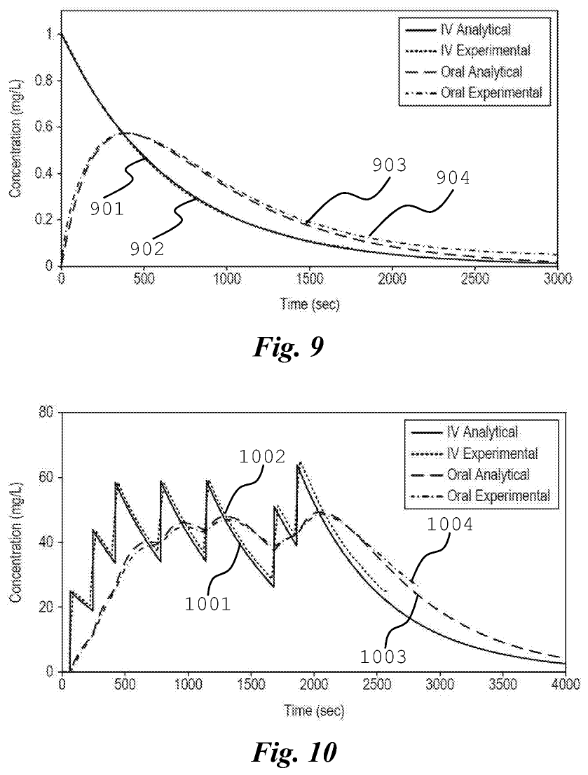

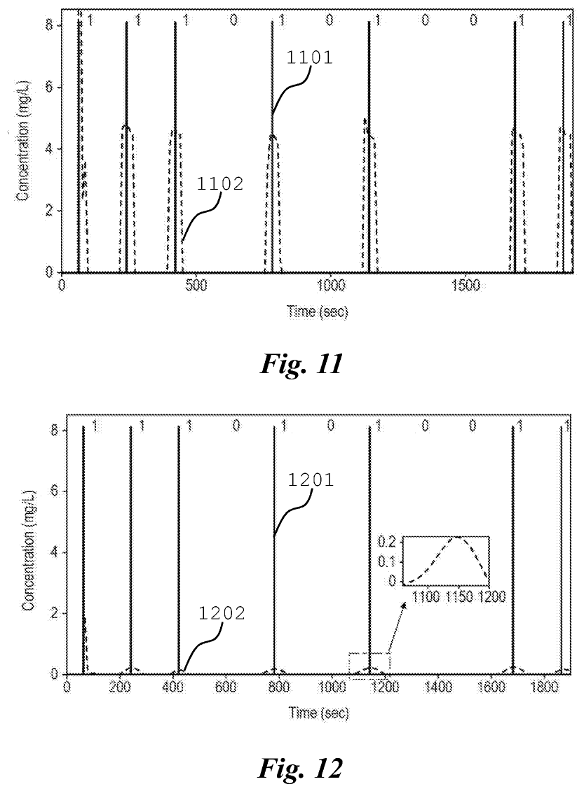

One way of inferring the information sent by the transmitter is to do demodulation of the sampled signal using deconvolution. In deconvolution, the receiver deconvolutes the sampled signal with the pre-generated impulse response curves of the body such as the one shown in FIG. 9. The result of deconvolution generates an approximation to the signal modulated by the transmitter as shown in FIGS. 11 and 12 which the receiver uses to detect symbols encoded within the signal and converts it to its equivalent binary bits and maps them to its equivalent message.

FIG. 8 is a diagram showing an example signaling molecules doses that generates a molecular signal (MS) for modulating a stream of bits (11101010011) according to an embodiment of the present invention. In this example, the transmitter is using On Off Keying where it injects signaling molecules to modulate a one and stop injecting to modulate a zero.

FIG. 9 is a diagram showing an example of the analytical (901,903) and experimental (902,904) normalized impulse response for the intravenous (901,902) and extravascular (903,904) administration according to an embodiment of the present invention. The analytical intravenous impulse response 901 is generated using the following equation:

$${C}_{B} = \frac{{B}_{0}}{V} {e}^{- {k}_{e} t}$$

where B₀ is total amount of signaling molecules injected into the body at t=0, V is the apparent volume of distribution and kₑ is the first order elimination rate constant for the signaling molecules. Similarly, the analytical extravascular impulse response 903 is generated using the following equation:

$${C}_{B} = \frac{{k}_{a} F {A}_{0}}{V ( {k}_{a} - {k}_{e} )} ( {e}^{- {k}_{e}} - {e}^{- {k}_{a} t} )$$

where A₀ is total amount of signaling molecules injected into the body at t=0, F is the absorbable fraction of A₀, V is the apparent volume of distribution, kₑ and kₑ are the first order elimination and absorption rate constants for the signaling molecules.

The experimental impulse responses (902,904) are found using an experimental platform that uses compartments that mimic the body environment. To generate the impulse response experimentally, a dose of signaling molecules is designed to be an input signal which is created to resemble a delta signal that has infinite magnitude and zero width. To reduce the width of the signal, the dose is put very quickly in a one shot manner to generate the required impulse effect. Then, the concentration of signaling molecules is measured at regular intervals. At the end of the experiment, the measurements are normalized which results in the impulse response for intravenous administration 902 and the impulse response for the extravascular administration 904.

FIG. 10 is a diagram showing an example of an analytical (1001,1003) and an experimental (1002,1004) MS wave in body captured by a receiver for intravenous (1001,1002) and extravascular (1003,1004) administrations according to an embodiment of the present invention. The analytical signals (1001,1003) are generated by convoluting the input signal shown in FIG. 8 with the corresponding impulse responses for the intravenous FIG. 9 (901) and extravascular FIG. 9 (903) cases. The experimental signals (1002,1004) are found using an experimental platform that uses compartments that mimic the body environment. The transmitter uses a pump to inject signaling molecules in the experimental platform using On Off Keying corresponding to the input signal of FIG. 8. At the same time the receiver is measuring the concentration of the signaling molecules on regular intervals. The measurements are plotted in the intravenous 1002 and extravascular 1004 administrations.

FIG. 11 is a diagram showing an example of an analytical 1101 and an experimental 1102 recovered input signal at the receiver for intravenous administrations according to an embodiment of the present invention. The analytical 1101 and experimental 1102 recovered input signals are found by deconvoluting the analytical FIG. 10 (1001) and experimental FIG. 10 (1002) intravenous signals with the corresponding impulse response curves in FIG. 9 (901,902). The receiver uses the recovered input signal to detect symbols encoded within the signal and converts it to its equivalent binary bits.

FIG. 12 is a diagram showing an example of an analytical 1201 and experimental 1202 recovered input signal at the receiver for extravascular administrations according to an embodiment of the present invention. The analytical 1201 and experimental 1202 recovered input signals are found by deconvoluting the analytical FIG. 10 (1003) and experimental FIG. 10 (1004) intravenous signals with the corresponding impulse response curves in FIG. 9 (903,904).

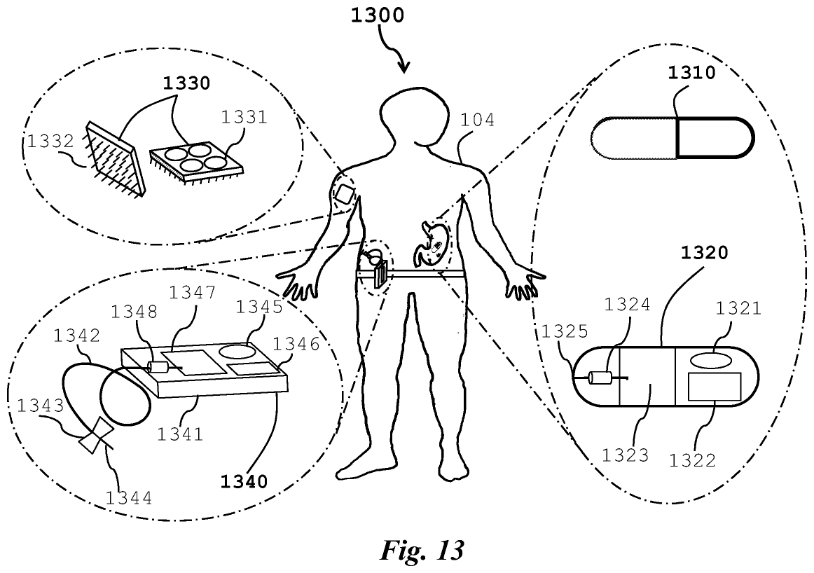

FIG. 13 is a diagram showing examples of different types of MoCoBo transmitters 1300. There are many routes a molecular signal can take into the body, such as but not limited to, oral, dermal, intravenous, or inhaling. The molecular signal route of administration determines the MoCoBo transmitter form. For example, in oral administration the transmitter components can be enclosed in a pill shaped container so that they can be taken orally and work from inside the body as shown in FIGS. 13 (1310) and (1320). Alternatively, the transmitter could be designed to work from out of the body by attaching it to the body from outside such as transmitters in FIGS. 13 (1330) and (1340).

FIG. 13 (1310) shows an example of a passive 2-bits smart pill that can modulate two bits and can be taken orally. The pill in this example contains two compartments denoted C₁ and C₂, but more or less number of compartments can be added without loss of generality. Each compartment may contain a solid or liquid form of a signaling molecules or combination of both. The compartments may be made from membranes or materials that has different biodegradability or made from the same material with different thickness so that the dissolution time of one compartment is different than the other. For example, the dissolution time T₁ of C₁ is greater than the dissolution time T₂ of C₂. To modulate two bits of information, one can use two levels of concentrations of signaling molecules, denoted L₀ and L₁. By setting different combinations of L₀ and L₁ in the compartments, a MS corresponding to 00, 01, 10, and 11 can be generated similar to the one shown in FIG. 4.

FIG. 13 (1320) shows an active transmitter in the form of a pill that can be taken orally and work from inside the body. The transmitter may comprise a microcontroller 1321, power source 1322, signaling molecules reservoir 1323, a releasing mechanism 1324, and an aperture 1325. The microcontroller 1321 is powered by the power source 1322 and is programmed to perform all the functions of a traditional transmitter except that it has a mechanism to control the signaling molecules amount and release time based on the information to be modulated. In this example, the release mechanism could be controlled using a pump 1324 that is powered to administer molecules through the aperture 1325 and switched off to stop.

FIG. 13 (1330) shows a passive transmitter that could be realized in the form of a patch that has compartments 1331 for storing the signaling molecules and controlling their release time using material that has different dissolution time such as biodegradable material. The molecules would be carried from the compartments and injected into the body via micro needles 1332. In this example, a passive 4-bits smart patch is shown.

FIG. 13 (1340) shows an active transmitter that could be placed inside a small box 1341 with an infusion set 1342 that has a cannula 1344. The cannula 1344 is inserted through the skin into the body and a patch 1343 which is used to hold the cannula in place while delivering the signaling molecules to the body. The transmitter may comprise a microcontroller 1345, power source 1346, signaling molecules reservoir 1347 and a releasing mechanism 1348. The microcontroller 1345 is powered by the power source 1346 and is programmed to perform all the functions of a traditional transmitter except that it has a mechanism to control the signaling molecules amount and release time based on the information to be modulated. In this example, the release mechanism could be controlled using a pump 1347 that is powered to administer molecules and switched off to stop.

What is claimed is:

1-20. (canceled)

21. A method of molecular communication within a body of an organism with a circulatory system through a molecular signaling method (MoSiMe), the method comprising the steps of:
providing a molecular communication system in body (MoCoBo) comprising a signaling molecule, a transmitter, a receiver, and a body of an organism with the circulatory system in which the molecule is released from a transmitter to a receiver;
modulating information by releasing the signaling molecule by the transmitter into the body of the organism;
detecting the change in the number of the signaling molecule by the receiver;
varying the number of the signaling molecule injected into the body of an organism to modulate a different symbol; and
waiting for a period of time between modulating symbols to allow the receiver to distinguish symbols from one another.

22. The method of claim 1, wherein the transmitter controls the amount and release time of signaling molecules using one or more parameters of the absorption, distribution, metabolism, and excretion (ADME) processes to inject them into the body for creating a MoSiMe signal enough for the receiver to detect and extract an encoded information within it.

23. The method of claim 1, wherein the transmitter uses more than one type of signaling molecule to transmit more than one symbol at the same time.

24. The method of claim 1, wherein the step of modulating information by releasing the signaling molecule by the transmitter into the body of the organism consists of injecting molecules into the body before modulating information to signal the start of transmission or wake up mode in the receiver.

25. The method of claim 1, wherein the step of modulating information by releasing the signaling molecule by the transmitter into the body of the organism consists of injecting the signaling molecule into the body after modulating information to signal the end of transmission or sleep mode in the receiver.

26. The method of claim 1, wherein the step of varying the number of molecules injected into the body of the organism to modulate different symbols consists of injecting no molecule to modulate a symbol.

27. The method of claim 1, wherein the step of waiting for a period of time between modulating symbols consists of omitting the waiting time between some of the symbols.

28. The method of claim 1, wherein the step of detecting the change in the number of signaling molecule by the receiver consists of measuring changes in a voltage due to changes in the signaling molecule quantity present in the body.

29. The method of claim 1, wherein the step of detecting the change in the number of molecules by the receiver consists of measuring changes in electrical current due to changes in the signaling molecule quantity present in the body.

30. The method of claim 1, wherein the step of detecting the change in the number of signaling molecule by the receiver consists of measuring the concentration of signaling molecules by the receiver.

31. The method of claim 1, wherein the step of detecting the change in the number of signaling molecules by the receiver consists of demodulating the changes in the number of signaling molecules over a period of time to their corresponding symbols.

32. The method of claim 1, wherein the receiver is a plurality of receivers.

33. The method of claim 1, wherein the receiver is a plurality of transmitters.

34. The method of claim 1, wherein the body of an organism is a human body.

35. The method of claim 1, wherein the body of an organism is an animal body.

36. The method of claim 1, wherein the signaling molecule is a plurality of molecules.
